(19) United States
(12) Patent Application Publication
(10) Pub. No.: US 2025/0235287 A1
(43) Pub. Date: Jul. 24, 2025

(54) COMPUTER-ASSISTED DISTANCE MEASUREMENT IN A SURGICAL SPACE

(71) Applicant: Intuitive Surgical Operations, Inc., Sunnyvale, CA (US)

(72) Inventors: Wenqing SUN, Mountain View, CA (US); Maximilian H. ALLAN, Mill Valley, CA (US); Kennneth K. LEE, Oakton, VA (US); A. Jonathan MCLEOD, Sunnyvale, CA (US)

(21) Appl. No.: 19/014,355

(22) Filed: Jan. 9, 2025

Related U.S. Application Data
(60) Provisional application No. 63/623,437, filed on Jan. 22, 2024.

(51) Int. Cl. A61B 90/00

(52) U.S. Cl. CPC A61B 90/361; A61B 90/06; A61B 2090/061

(57) ABSTRACT

This disclosure describes a system for measuring a distance between two points in a surgical space. The system moves an imaging device to a first pose to capture first imaging data showing a first region of a surface of an anatomical structure, receives first imaging data, determines a first distance between the imaging device in the first pose and a first point on the surface, and determines first coordinates of the first point. The system moves the imaging device to a second pose to capture second imaging data showing a second region of the surface, receives second imaging data, determines a second distance between the imaging device in the second pose and a second point on the surface, and determines second coordinates for the second point. The system determines, based on the first coordinates and the second coordinates, a third distance between the first point and the second point.

### RELATED APPLICATION

This application claims the benefit of U.S. provisional patent application Ser. No. 63/623,437, filed Jan. 22, 2024, which is hereby incorporated herein by reference.

### TECHNICAL FIELD

The present disclosure relates generally to medical systems. Specifically, the present disclosure relates to using a medical system to measure a distance in a surgical space.

### BACKGROUND

Various surgical instruments are introduced in a surgical space to perform operations on patients. Such surgical instruments can include cameras. In one example, stereo cameras are used. Stereo cameras include two cameras (e.g., a left camera and a right camera). A triangulation technique may be employed to determine a point in 3D space given the point's projections onto images from the left camera and the right camera, which provides a depth measurement for the point. The triangulation technique relies on a baseline (center point) distance between the left camera and the right camera to determine the depth of the point. The baseline distance between the left camera and the right camera may be small, which results in the determined depth being more inaccurate the further away the point is from the stereo camera.

### SUMMARY

The present disclosure describes a system and method for measuring a distance between two points in a surgical space. According to an embodiment, a system for measuring a distance between two points in a surgical space includes a memory and a controller communicatively coupled to the memory. The system moves an imaging device to a first pose to capture first imaging data showing a first region of a surface of an anatomical structure, determines a first distance between the imaging device in the first pose and a first point on the surface, and determines, based on the first pose and the first distance, first coordinates of the first point on the surface. The system also moves the imaging device to a second pose to capture second imaging data showing a second region of the surface of the anatomical structure, determines a second distance between the imaging device in the second pose and a second point on the surface, and determines, based on the second pose and the second distance, second coordinates for the second point on the surface. The system, based on each of the first and second distances to the first and the second points on the surface, respectively, being within a threshold, determines, based on the first coordinates and the second coordinates, a third distance between the first point and the second point.

According to another embodiment, a method for measuring a distance between two points in a surgical space includes moving an imaging device to a first pose to capture first imaging data showing a first region of a surface of an anatomical structure, determining a first distance between the imaging device in the first pose and a first point on the surface, and determining, based on the first pose and the first distance, first coordinates of the first point on the surface. The method further includes moving the imaging device to a second pose to capture second imaging data showing a second region of the surface of the anatomical structure, determining a second distance between the imaging device in the second pose and a second point on the surface, and determining, based on the second pose and the second distance, second coordinates for the second point on the surface. The method further includes, based on each of the first and second distances to the first and the second points on the surface, respectively, being within a threshold, determining, based on the first coordinates and the second coordinates, a third distance between the first point and the second point. Other embodiments includes a non-transitory machine-readable medium storing instructions that, when executed by a processor, cause the processor to perform the method.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1A illustrates an example medical system.

FIGS. 1B and 1C illustrate example components in the system of FIG. 1A.

FIG. 2A illustrates an example medical system.

FIGS. 2B and 2C illustrate an example medical instrument system in the system of FIG. 2A.

FIGS. 3A through 3C illustrate example operations performed by the system of FIG. 1A or 2A.

FIG. 4 is a flowchart of an example method performed by the system of FIG. 1A or 2A.

FIG. 5 illustrates an example operation performed by the system of FIG. 1A or 2A.

FIGS. 6A through 6C illustrate example operations performed by the system of FIG. 1A or 2A.

FIG. 7 is a flowchart of an example method performed by the system of FIG. 1A or 2A.

FIG. 8 is a flowchart of an example method performed by the system of FIG. 1A or 2A.

FIG. 9 illustrates an example operation performed by the system of FIG. 1A or 2A.

FIG. 10 illustrates an example operation performed by the system of FIG. 1A or 2A.

FIG. 11 is a flowchart of an example method performed by the system of FIG. 1A or 2A.

FIG. 12 illustrates an example operation performed by the system of FIG. 1A or 2A.

FIG. 13 illustrates an example operation performed by the system of FIG. 1A or 2A.

### DETAILED DESCRIPTION

Endoscopy is a medical procedure to visualize the human body's internal organs or natural cavities. The endoscope is a rigid or flexible tubular device allowing a direct view into the body. An endoscopic system can be built up as a purely optical device containing lenses, transparent rods or fibers, or in combination with integrated or add-on cameras. The endoscope tip can be inserted through a small access created by incision or through the natural lumina of the body. A light source on the endoscope provides sufficient illumination to the examined cavity. Cameras on the endoscope provide a field of view that can be visualized on a screen and recorded for later diagnosis or documentation. Two cameras (e.g., a left camera and a right camera) can be placed side-by-side on the endoscope to form a stereo camera, which provides a stereo view and allows for depth perception. For example, the two cameras may capture images from slightly different perspectives, similar to how human eyes perceive depth. These dual images are then processed to create a 3D image or video, providing surgeons and medical professionals with improved spatial awareness during procedures. For example, a triangulation technique may be performed using the images from the two cameras to determine the distances between different points in the view and the camera (e.g., the depths of the points) and the distances between these points, which allows surgeons to better understand the relative distances between different structures or instruments in the view. This enhanced spatial awareness may be beneficial for performing delicate and precise maneuvers.

The triangulation technique uses a baseline (center point) distance between the left camera and the right camera and the difference in the position of a first point between images from the left camera and the right camera to determine the position of the first point and the distance between the first point and the endoscope. This process may be repeated for a second point in the view to determine the position of the second point and the distance between the second point and the endoscope. The distance between the first and second points may then be determined using the positions of the first and second points and the distances between the first and second points and the endoscope.

The baseline distance between the left camera and the right camera may be small (e.g., 4 millimeters), which results in the determined distances between the first and second points and the endoscope being more inaccurate the further away the first and second points are from the stereo camera in the endoscope. Moving the stereo camera closer to the first point or the second point may improve the accuracy of the distance measurement, however, it may also cause the other point to fall out of view, which may prevent the triangulation technique from being used for the other point. As a result, it becomes more difficult to accurately determine the distance between the two points the further away the two points are from each other.

The present disclosure describes a medical system that measures a distance between two points in a surgical space by leveraging kinematics data that describes the pose (e.g., position and orientation) of an endoscope. Generally, the system adjusts the pose of the endoscope so that the endoscope is close to a first point to triangulate the distance between the first point and the endoscope. The kinematics data indicates the pose of the endoscope, and so the kinematics data may be used with the determined distance between the first point and the endoscope to determine global coordinates for the first point. The system then adjusts the pose of the endoscope so that the endoscope is close to a second point to triangulate the distance between the second point and the endoscope. The kinematics data indicates the new pose of the endoscope, and so the kinematics data may be used with the determined distance between the second point and the endoscope to determine global coordinates for the second point. Adjusting the pose of the endoscope so that the endoscope is close to the second point may cause the first point to fall out of view. Nevertheless, because the kinematics data indicates the poses of the endoscope, it is possible to use the kinematics data to determine the global coordinates of the first point and the second point. These global coordinates are then used to determine a distance between the first point and the second point.

In certain embodiments, the system provides several technical advantages. For example, the system may provide a more accurate distance measurement between two points in a surgical space. Specifically, the system may bring the endoscope close to the two points to triangulate the depths and/or positions of the two points, which improves the accuracy of the triangulation. The system uses the kinematics data to track the poses of the endoscope, which allows the system to determine the distance between the two points using the determined depths and/or positions of the two points, even if bringing the endoscope close to one of the points causes the other point to fall out of view. The more accurate measurements of the distance between the two points allow a surgeon to precisely locate anatomical structures, lesions, or target areas within the patient's body. This precision is important for performing delicate and targeted surgical procedures, which minimizes errors. Surgeons can also rely on the information to navigate through complex anatomical structures and to avoid unintentional damage to surrounding tissues. Surgeons can use this information to plan and execute procedures with a high level of confidence, ensuring that critical structures are identified and treated appropriately. More accurate spatial information also helps guide the placement of instruments and allows for effective navigation through narrow and confined spaces, which contributes to the efficient use of surgical resources, including time and equipment. Thus, the system may improve the health and safety of a patient and reduce recovery times and postoperative complications.

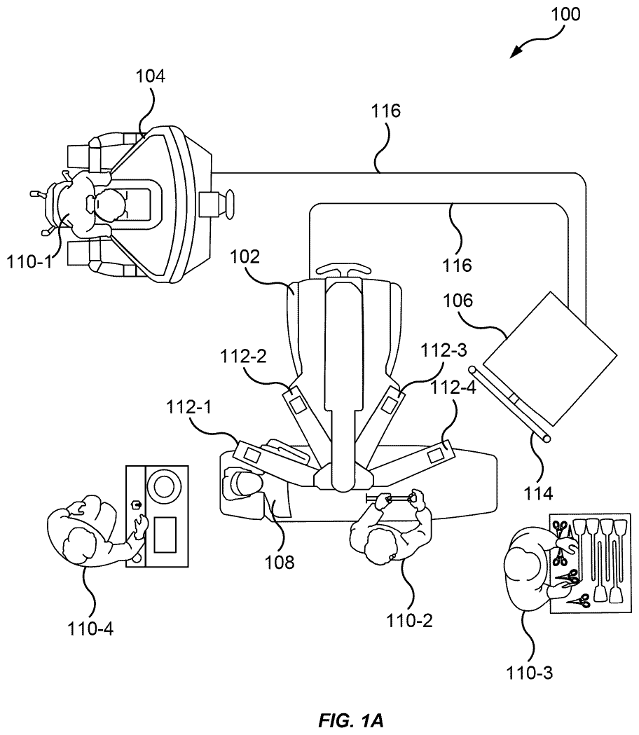

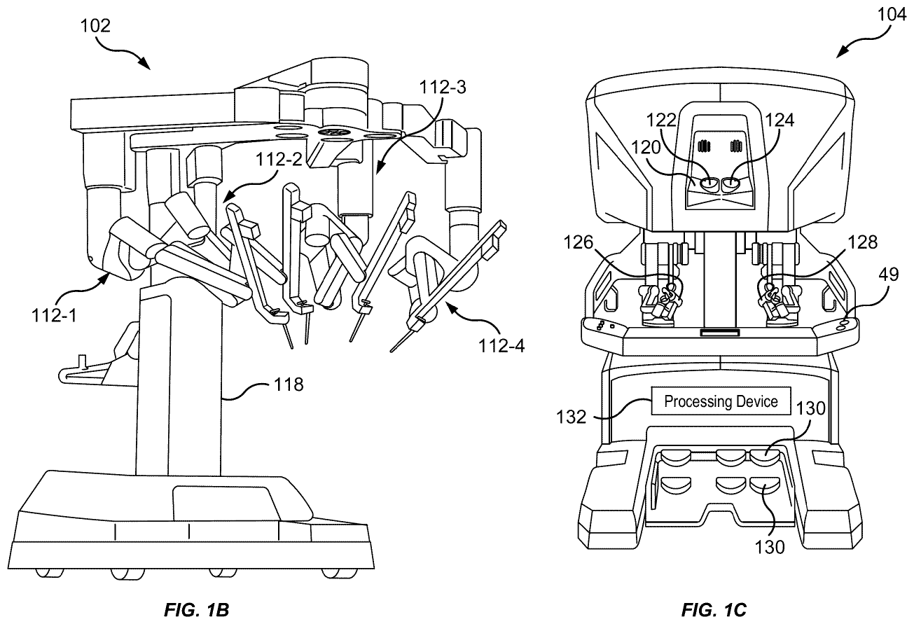

In some examples, one or more components of a medical system may be implemented as a computer-assisted surgical system. It is understood, however, that the medical system may be implemented in any type of medical system (e.g., digital fiducial systems, anatomy detection systems, and clinical guidance systems). FIG. 1A shows an example computer-assisted surgical system 100 that may implement some of the features described herein.

The surgical system 100 includes a manipulator assembly 102, a user control apparatus 104, and an auxiliary apparatus 106, all of which are communicatively coupled to each other. The surgical system 100 is utilized by a medical team to perform a computer-assisted medical procedure or other similar operation on a body of a patient 108 or on any other body as may serve a particular implementation. The medical team includes a first user 110-1 (such as a surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom are collectively referred to as users 110, and each of whom may control, interact with, or otherwise be a user of the surgical system 100. More, fewer, or alternative users may be present during a medical procedure as may serve a particular implementation. For example, team composition for different medical procedures, or for non-medical procedures, may differ and include users with different roles.

Although FIG. 1A illustrates an ongoing minimally invasive medical procedure such as a minimally invasive surgical procedure, it will be understood that the surgical system 100 may similarly be used to perform open medical procedures or other types of operations. For example, operations such as exploratory imaging operations, mock medical procedures used for training purposes, and/or other operations may also be performed.

The manipulator assembly 102 includes one or more manipulator arms 112 (e.g., manipulator arms 112-1 through 112-4) to which one or more instruments may be coupled. The instruments are used for a computer-assisted surgical procedure on the patient 108 (e.g., by being at least partially inserted into the patient 108 and manipulated within the patient 108). While the manipulator assembly 102 is depicted and described herein as including four manipulator arms 112, the manipulator assembly 102 may include a single manipulator arm 112 or any other number of manipulator arms as may serve a particular implementation. Although the example of FIG. 1 illustrates the manipulator arms 112 as robotic manipulator arms, one or more instruments may be partially or entirely manually controlled, such as by being handheld and controlled manually by a person. These partially or entirely manually controlled instruments are used in conjunction with, or as an alternative to, computer-assisted instrumentation that is coupled to the manipulator arms 112.

During the medical operation, the user control apparatus 104 facilitates tele-operational control by the user 110-1 of the manipulator arms 112 and instruments attached to the manipulator arms 112. To this end, the user control apparatus 104 provides the user 110-1 with imagery of an operational area associated with the patient 108 as captured by an imaging device. The manipulator arms 112 or any instruments coupled to the manipulator arms 112 mimic the dexterity of the hand, wrist, and fingers of the user 110-1 across multiple degrees of freedom of motion. In this manner, the user 110-1 intuitively performs a procedure (e.g., an incision procedure, a suturing procedure, etc.) using one or more of the manipulator arms 112 or any instruments coupled to the manipulator arms 112.

The auxiliary apparatus 106 includes one or more computing devices that perform auxiliary functions in support of the procedure, such as providing insufflation, electrocautery energy, illumination or other energy for imaging devices, image processing, or coordinating components of the surgical system 100. The auxiliary apparatus 106 includes a display monitor 114 that displays one or more user interfaces, or graphical or textual information in support of the procedure. In some instances, the display monitor 114 is a touchscreen display that provides user input functionality. Augmented content provided by a region-based augmentation system may be similar to, or differ from, content associated with the display monitor 114 or one or more display devices in the operation area (not shown).

The manipulator assembly 102, user control apparatus 104, and auxiliary apparatus 106 are communicatively coupled one to another in any suitable manner. The manipulator assembly 102, user control apparatus 104, and auxiliary apparatus 106 may be communicatively coupled by way of control lines 116, which represent any wired or wireless communication link as may serve a particular implementation. To this end, the manipulator assembly 102, user control apparatus 104, and auxiliary apparatus 106 may each include one or more wired or wireless communication interfaces, such as one or more local area network interfaces, Wi-Fi network interfaces, cellular interfaces, and so forth.

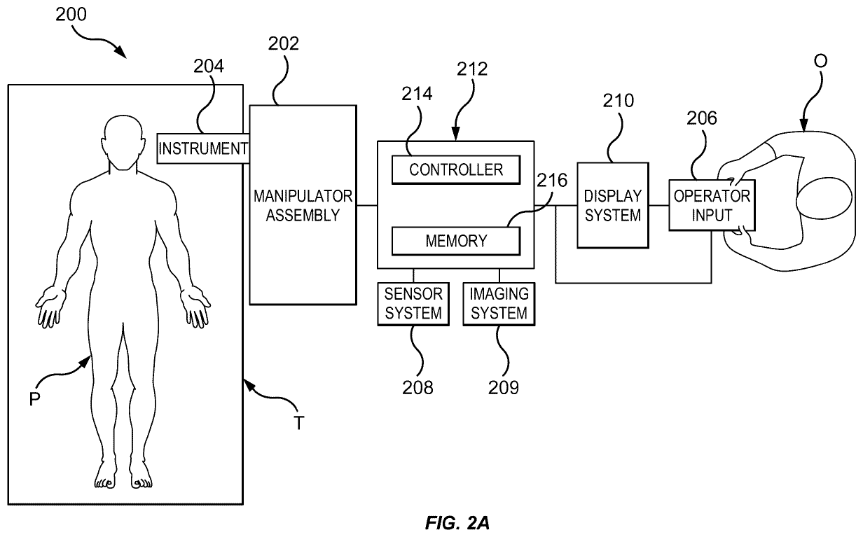

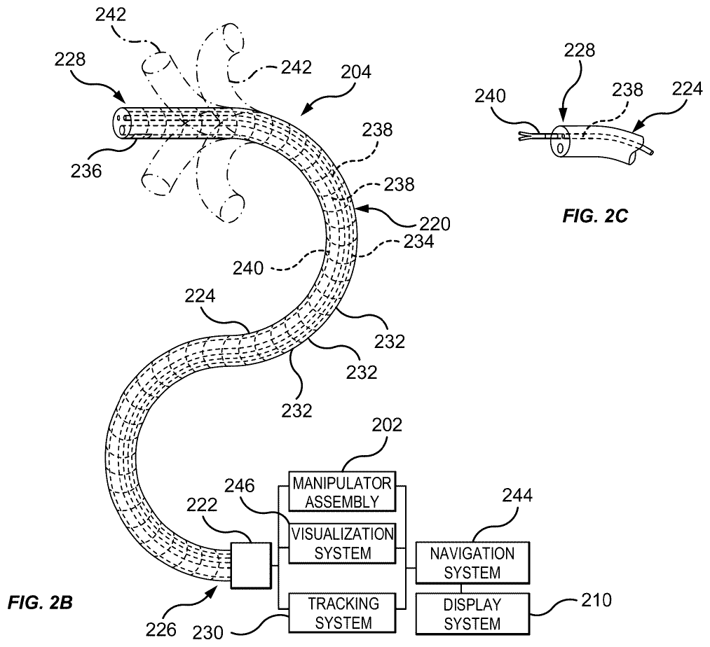

FIG. 1B illustrates an example manipulator assembly 102. As seen in FIG. 2A, the manipulator assembly 102 includes a base 118, a manipulator arm 112-1, a manipulator arm 112-2, a manipulator arm 112-3, and a manipulator arm 112-4. Each manipulator arm 112-1, 112-2, 112-3, and 112-4 is pivotably coupled to the base 118. Although the base 118 may include casters to allow ease of mobility, in some embodiments, the manipulator assembly 102 is fixedly mounted to a floor, ceiling, operating table, structural framework, or the like.

In a typical procedure, two of the manipulator arms 112-1, 112-2, 112-3, or 112-4 hold surgical instruments and a third holds a stereo endoscope. The remaining manipulator arms are available so that other instruments may be introduced at the work site. Alternatively, the remaining manipulator arms may be used for introducing another endoscope or another image capturing device, such as an ultrasound transducer, to the work site.

Each of the manipulator arms 112-1, 112-2, 112-3, and 112-4 are formed of links that are coupled together and manipulated through actuatable joints. Each of the manipulator arms 112-1, 112-2, 112-3, and 112-4 may include a setup arm and a device manipulator. The setup arm positions its held device so that a pivot point occurs at its entry aperture into the patient. The device manipulator may then manipulate its held device so that the held device may be pivoted about the pivot point, inserted into and retracted out of the entry aperture, and rotated about its shaft axis. Each of the manipulator arms 112-1, 112-2, 112-3, and 112-4 may include sensors (e.g., joint sensors, position sensors, accelerometers, etc.) that detect or track movement of the manipulator arms 112-1, 112-2, 112-3, and 112-4. For example, these sensors may detect how far or how quickly a manipulator arm 112-1, 112-2, 112-3, or 112-4 moves in a certain direction.

FIG. 1C illustrates an example user control apparatus 104. The user control apparatus 104 includes a stereo vision display 120 so that the user may view the surgical work site in stereo vision from images captured by the stereoscopic camera of the manipulator assembly 102. Left and right eyepieces 122 and 124 are provided in the stereo vision display 120 so that the user may view left and right display screens inside the display 120 respectively with the user's left and right eyes. While viewing typically an image of the surgical site on a suitable viewer or display, the surgeon performs the surgical procedures on the patient by manipulating master control input devices, which in turn control the motion of robotic instruments.

The user control apparatus 104 also includes left and right input devices 126 and 128 that the user grasps respectively with his/her left and right hands to manipulate devices (e.g., surgical instruments) being held by the manipulator arms 112-1, 112-2, 112-3, and 112-3 of the manipulator assembly 102 in preferably six or more degrees of freedom (“DOF”). Foot pedals 130 with toe and heel controls are provided on the user control apparatus 104 so the user may control movement and/or actuation of devices associated with the foot pedals.

A processing device 132 is provided in the user control apparatus 104 for control and other purposes. The processing device 132 performs various functions in the surgical system 100. One function performed by processing device 132 is to translate and transfer the mechanical motion of input devices 126 and 128 to actuate their corresponding joints in their associated manipulator arms 112-1, 112-2, 112-3, and 112-4 so that the surgeon can effectively manipulate devices, such as the surgical instruments. Another function of the processing device 132 is to implement the methods, crosscoupling control logic, and controllers or processors described herein. The auxiliary apparatus 106 may include a processing device 132 that performs the functions or actions described herein. The processing device 132 includes a controller and a memory that perform the functions described herein. The controller may include one or more processors.

The controller may include any electronic circuitry, including, but not limited to one or a combination of microprocessors, microcontrollers, application specific integrated circuits (ASIC), application specific instruction set processor (ASIP), and/or state machines, that communicatively couples to a memory and controls the operation of the user control apparatus 104 and/or the auxiliary apparatus 106. The controller may be 8-bit, 16-bit, 32-bit, 64-bit or of any other suitable architecture. The controller may include an arithmetic logic unit (ALU) for performing arithmetic and logic operations, registers that supply operands to the ALU and store the results of ALU operations, and a control unit that fetches instructions from memory and executes them by directing the coordinated operations of the ALU, registers and other components. The controller may include other hardware that operates software to control and process information. The controller executes software stored on a memory to perform any of the functions described herein. The controller controls the operation and administration of the user control apparatus 104 or the auxiliary apparatus 106 by processing information (e.g., information received from the user control apparatus 104, the manipulator assembly 102, the auxiliary apparatus 106, and/or a memory). The controller is not limited to a single processing device and may encompass multiple processing devices contained in the same device or computer or distributed across multiple devices or computers. The controller is considered to perform a set of functions or actions if the multiple processing devices collectively perform the set of functions or actions, even if different processing devices perform different functions or actions in the set.

FIG. 2A illustrates an example computer-assisted surgical system 200 that implements some of the features described herein. The surgical system 200 can be used, for example, in surgical, diagnostic, therapeutic, biopsy, or non-medical procedures. As shown in FIG. 2A, the surgical system 200 (which may be a robotically-assisted surgical system) includes one or more manipulator assemblies 202 for operating one or more medical instrument systems 204 in performing various procedures on a patient P positioned on a table T in a medical environment. For example, the manipulator assembly 202 can drive catheter or end effector motion, can apply treatment to target tissue, and/or can manipulate control members. The manipulator assembly 202 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 206, which can be inside or outside of the medical environment, generally includes one or more control devices for controlling the manipulator assembly 202. The manipulator assembly 202 supports a medical instrument system 204 and can optionally include a plurality of actuators or motors that drive inputs on the medical instrument system 204 in response to commands from a control system 212. The actuators can optionally include drive systems that when coupled to the medical instrument system 204 can advance the medical instrument system 204 into a natural or surgically created anatomic orifice. Other drive systems can move the distal end of the medical instrument in multiple degrees of freedom, which can include three degrees of linear motion (e.g., linear motion along the x, y, and z Cartesian axes) and in three degrees of rotational motion (e.g., rotation about the x, y, and z Cartesian axes). The manipulator assembly 202 can support various other systems for irrigation, treatment, or other purposes. Such systems can include fluid systems (e.g., reservoirs, heating/cooling elements, pumps, and valves), generators, lasers, interrogators, and ablation components.

The surgical system 200 also includes a display system 210 for displaying an image or representation of the surgical site and a medical instrument system 204. The image or representation is generated by an imaging system 209, which may include an endoscopic imaging system. The display system 210 and operator input system 206 may be oriented so that an operator O can control the medical instrument system 204 and the operator input system 206 with the perception of telepresence. A graphical user interface can be displayable on the display system 210 and/or a display system of an independent planning workstation.

In some examples, the imaging system 209 includes an endoscopic imaging system with components that are integrally or removably coupled to the medical instrument system 204. However, in some examples, a separate imaging device, such as an endoscope, attached to a separate manipulator assembly can be used with the medical instrument system 204 to image the surgical site. The imaging system 209 can be implemented as hardware, firmware, software, or a combination thereof, which interact with or are otherwise executed by one or more computer processors, which can include the controller 214 of the control system 212.

The surgical system 200 also includes a sensor system 208. The sensor system 208 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 204. These sensors may also detect a position, orientation, or pose of the patient P on the table T. For example, the sensors may detect whether the patient P is face-down or face-up. As another example, the sensors may detect a direction in which the head of the patient P is directed. The sensor system 208 can also include temperature, pressure, force, or contact sensors, or the like.

The surgical system 200 can also include a control system 212, which includes at least one memory 216 and at least one controller 214 (which may include a processor) for effecting control between the medical instrument system 204, the operator input system 206, the sensor system 208, and the display system 210. The control system 212 includes programmed instructions (e.g., a non-transitory machine-readable medium storing the instructions) to implement a procedure using the surgical system 200, including for navigation, steering, imaging, engagement feature deployment or retraction, applying treatment to target tissue (e.g., via the application of energy), or the like.

The control system 212 may further include a virtual visualization system to provide navigation assistance to the operator O when controlling medical instrument system 204 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology, such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 212 uses a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

The controller 214 is any electronic circuitry, including, but not limited to one or a combination of microprocessors, microcontrollers, application specific integrated circuits (ASIC), application specific instruction set processor (ASIP), and/or state machines, that communicatively couples to the memory 216 and controls the operation of the control system 212. The controller 214 may be 8-bit, 16-bit, 32-bit, 64-bit or of any other suitable architecture. The controller 214 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The controller 214 may include other hardware that operates software to control and process information. The controller 214 executes software stored on the memory 216 to perform any of the functions described herein. The controller 214 controls the operation and administration of the control system 212 by processing information (e.g., information received from the manipulator assembly 202, the operator input system 206, and the memory 216). The controller 214 is not limited to a single processing device and may encompass multiple processing devices contained in the same device or computer or distributed across multiple devices or computers. The controller 214 is considered to perform a set of functions or actions if the multiple processing devices collectively perform the set of functions or actions, even if different processing devices perform different functions or actions in the set.

The memory 216 may store, either permanently or temporarily, data, operational software, or other information for the controller 214. The memory 216 may include any one or a combination of volatile or non-volatile local or remote devices suitable for storing information. For example, the memory 216 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 216, a disk, a CD, or a flash drive. In particular embodiments, the software may include an application executable by the controller 214 to perform one or more of the functions described herein. The memory 216 is not limited to a single memory and may encompass multiple memories contained in the same device or computer or distributed across multiple devices or computers. The memory 216 is considered to store a set of data, operational software, or information if the multiple memories collectively store the set of data, operational software, or information, even if different memories store different portions of the data, operational software, or information in the set.

FIG. 2B illustrates an example medical instrument system 204 in the surgical system 200. In some embodiments, the medical instrument system 204 is used in an image-guided medical procedure. For example, the medical instrument system 204 may be used for non-teleoperational exploratory procedures or in procedures involving traditional manually operated medical instruments, such as endoscopy.

The medical instrument system 204 includes an elongate flexible device 220, such as a flexible catheter or endoscope (e.g., gastroscope, bronchoscope), coupled to a drive unit 222. The elongate flexible device 220 includes a flexible body 224 having a proximal end 226 and a distal end, or tip portion, 228. In some embodiments, the flexible body 224 has an approximately 14-20 millimeter outer diameter. Other flexible body outer diameters may be larger or smaller. The flexible body 224 has an appropriate length to reach certain portions of the anatomy, such as the lungs, sinuses, throat, or the upper or lower gastrointestional region, when the flexible body 224 is inserted into a patient's oral or nasal cavity.

The medical instrument system 204 includes a tracking system 230 for determining the position, orientation, speed, velocity, pose, and/or shape of the distal end 228 and/or of one or more segments 232 along the flexible body 224 using one or more sensors and/or imaging devices. The entire length of the flexible body 224, between the distal end 228 and the proximal end 226, is effectively divided into the segments 232. The tracking system 230 is implemented as hardware, firmware, software, or a combination thereof, which interact with or are otherwise executed by one or more computer processors, which may include the controller 214 of control system 212.

The tracking system 230 tracks distal the end 228 and/or one or more of the segments 232 using a shape sensor 234. In some embodiments, the tracking system 230 tracks the distal end 228 using a position sensor system 236, such as an electromagnetic (EM) sensor system. In some examples, the position sensor system 236 measures six degrees of freedom (e.g., three position coordinates x, y, and z and three orientation angles indicating pitch, yaw, and roll of a base point) or five degrees of freedom (e.g., three position coordinates x, y, and z and two orientation angles indicating pitch and yaw of a base point).

The flexible body 224 includes one or more channels 238 sized and shaped to receive one or more medical instruments 240. In some embodiments, the flexible body 224 includes two channels 238 for separate instruments 240, however, a different number of channels 238 can be provided. FIG. 2C illustrates an example portion of the medical instrument system 204 of FIG. 2B. As seen in FIG. 2C, the medical instrument 240 extends through the flexible body 224. In some embodiments, the medical instrument 240 can be used for procedures and aspects of procedures, such as surgery, biopsy, ablation, mapping, imaging, illumination, irrigation, or suction. The medical instrument 240 is deployed through the channel 238 of the flexible body 224 and is used at a target location within the anatomy. The medical instrument 240 includes, for example, image capture devices, biopsy instruments, ablation instruments, catheters, laser ablation fibers, and/or other surgical, diagnostic, or therapeutic tools. The medical tools include end effectors having a single working member such as a scalpel, a blunt blade, a lens, an optical fiber, an electrode, and/or the like. Other end effectors include, for example, forceps, graspers, balloons, needles, scissors, clip appliers, and/or the like. Other end effectors further include electrically activated end effectors such as electrosurgical electrodes, transducers, sensors, imaging devices, and/or the like. The medical instrument 240 is advanced from the opening of the channel 238 to perform the procedure and then retracted back into the channel when the procedure is complete. The medical instrument 240 is removed from the proximal end 226 of the flexible body 224 or from another optional instrument port (not shown) along the flexible body 224. The medical instrument 240 may be used with an image capture device (e.g., an endoscopic camera) also within the elongate flexible device 220. Alternatively, the medical instrument 240 may itself be the image capture device.

The medical instrument 240 additionally houses cables, linkages, or other actuation controls (not shown) that extend between the proximal and distal ends to controllably bend the distal end of the medical instrument 240. The flexible body 224 also houses cables, linkages, or other steering controls (not shown) that extend between the drive unit 222 and the distal end 228 to controllably bend the distal end 228 as shown, for example, by the broken dashed line depictions 242 of the distal end 228. In some examples, at least four cables are used to provide independent “up-down” steering to control a pitch motion of the distal end 228 and “left-right” steering to control a yaw motion of the distal end 228. In embodiments in which the medical instrument system 204 is actuated by a robotically-assisted assembly, the drive unit 222 can include drive inputs that removably couple to and receive power from drive elements, such as actuators, of the teleoperational assembly. In some embodiments, the medical instrument system 204 includes gripping features, manual actuators, or other components for manually controlling the motion of the medical instrument system 204. The information from the tracking system 230 can be sent to a navigation system 244, where the information is combined with information from the visualization system 246 and/or the preoperatively obtained models to provide the physician or other operator with real-time position information.

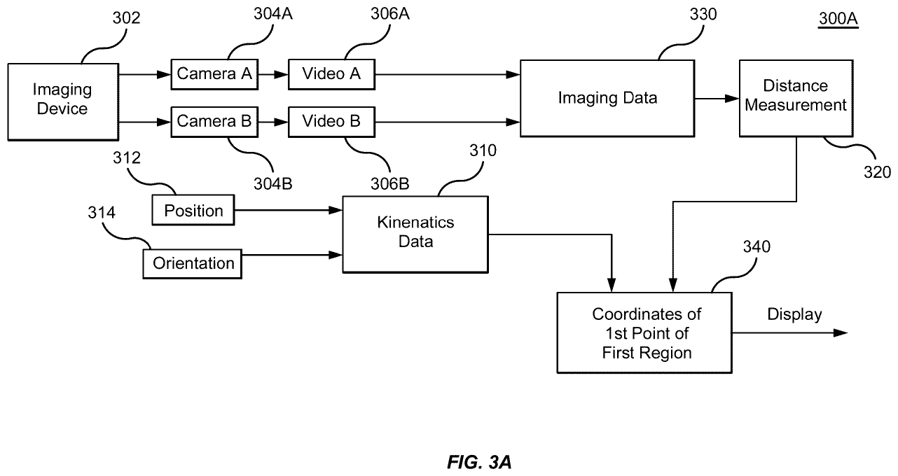

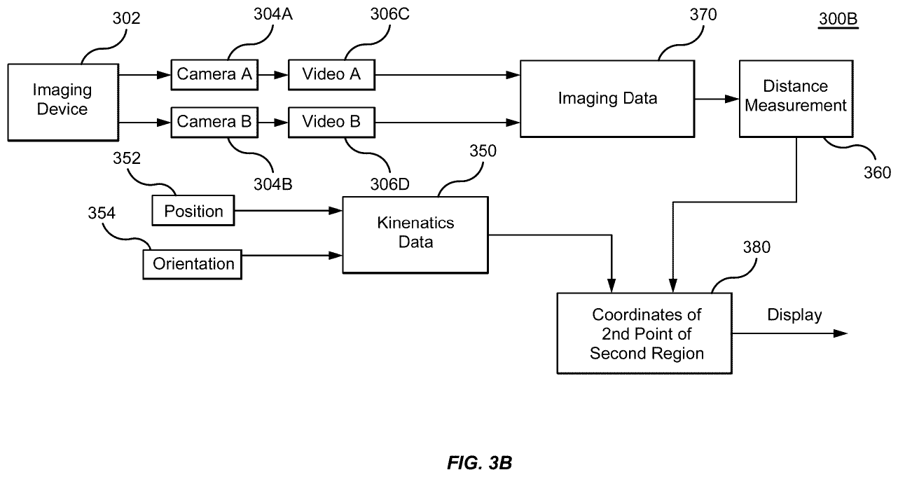

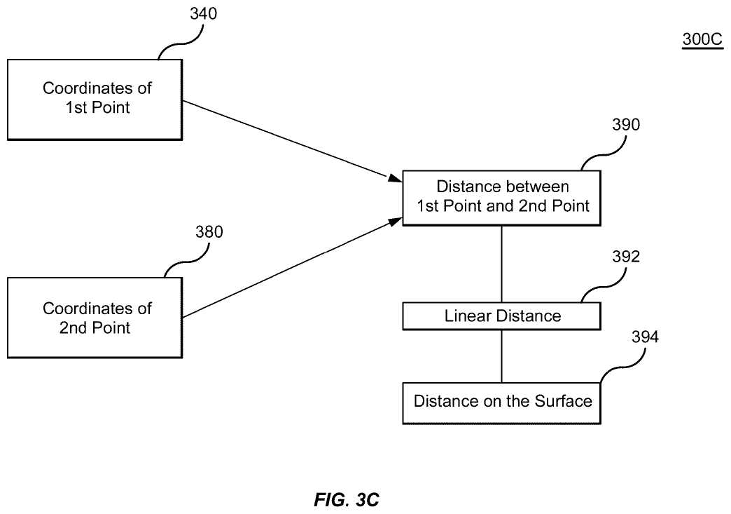

FIGS. 3A through 13 illustrate example operations performed by a computer system in a medical system (e.g., the surgical system 100 of FIG. 1A or the surgical system 200 of FIG. 2A). Generally, the computer system (which may be implemented in the user control apparatus 104 and/or the auxiliary apparatus 106 of the surgical system 100 using the processing device 132 and/or in the control system 212 of the surgical system 200 using the controller 214 and the memory 216) measures a distance between two points in a surgical space by leveraging kinematics data and depth data.

FIG. 3A illustrates an example operation 300A performed by the computer system. The computer system receives videos 306A and 306B captured by cameras 304A and 304B (e.g., a left camera and a right camera of a stereo camera) of an imaging device 302 (e.g., an endoscope). The cameras 304A and 304B are positioned at the front of the imaging device 302. The videos 306A and 306B may be captured from the perspective of the front of the imaging device 302. The imaging device 302 may be set at a first pose (e.g., position and orientation) such that the videos 306A and 306B show the surface of a first region of an anatomical structure (e.g., at a surgical site). Information from the videos 306A and 306B may form imaging data 330 of the first region of the anatomical structure.

The computer system monitors and tracks kinematics data 310 that indicates the first pose of the imaging device 302. The kinematics data 310 may include three positional degrees of freedom and three orientational degrees of freedom. The three positional degrees of freedom provide translation information of the imaging device 302 and indicate a position 312 of the imaging device 302. The three orientational degrees of freedom provide rotation information of the imaging device 302 and indicate an orientation 314 of the imaging device 302.

The computer system makes a distance measurement 320 from the imaging device 302 in the first pose to a first point on the first region of the anatomical structure. The distance measurement 320 may indicate a first distance between the imaging device 302 in the first pose and the first point. In some embodiments, the computer system uses triangulation to determine the distance measurement 320. For example, the computer system may be provided a distance between the camera 304A and the camera 304B (which may be referred to as a baseline or center point distance). The computer system then uses the imaging data 330 (which shows the first point) and the distance between the cameras 304A and 304B to triangulate the distance from the imaging device 302 to the first point.

In some embodiments, the computer system uses the video 306A from the camera 304A to determine a distance between the camera 304A and the first point. The computer system may then use the video 306B from the camera 304B to determine a distance between the camera 304B and the first point. The computer system then calculates a distance between the imaging device 302 and the first point using the distance between the camera 304A and the first point, the distance between the camera 304B and the first point, and the distance between the camera 304A and the camera 304B. For example, the computer system may calculate the distance between the imaging device 302 as the height of the triangle with sides having lengths equal to the distance between the camera 304A and the first point, the distance between the camera 304B and the first point, and the distance between the camera 304A and the camera 304B.

The computer system then uses the kinematics data 310, which indicates the position 312 and orientation 314 of the imaging device 302, along with the distance measurement 320 to determine coordinates 340 of the first point of the first region. For example, the position 312 may indicate the global coordinates of the imaging device 302. The orientation 314 may indicate a direction in which the imaging device 302 is pointed. The computer system may then determine the global coordinates for the first point by calculating the coordinate that is the distance measurement 320 away from the position 312 in the direction indicated by the orientation 314. These global coordinates for the first point may be the coordinates 340. The coordinates 340 are displayed on a display and may be expressed as Cartesian coordinates (x₁, y₁, z₁).

FIG. 3B illustrates an example operation 300B performed by the computer system. As seen in FIG. 3B, the imaging device 302 has been moved to a second pose. For example, the imaging device 302 may have been moved to a different position and/or rotated into a different orientation. Moreover, the imaging device 302 may be moved from the first pose to the second pose by a repositionable structure, such as the manipulator arms 112-1 through 112-4 described in FIG. 1A. The cameras 304A and 304B capture videos 306C and 306D of a second region of the surface of the anatomical structure when the imaging device 302 is in the second pose. The videos 306C and 306D form imaging data 370 of the second region of the anatomical structure.

The computer system monitors and tracks kinematics data 350 that indicates the second pose of the imaging device 302. The kinematics data 350 includes three positional degrees of freedom and three orientational degrees of freedom. The three positional degrees of freedom provide translation information of the imaging device 302 and indicate a position 352 of the imaging device 302. The three orientational degrees of freedom provide rotation information of the imaging device 302 and indicate an orientation 354 of the imaging device 302. The position 352 and the orientation 354 may be different from the position 312 and the orientation 314 of the imaging device 302 due to the movement of the imaging device 302 from the first pose to the second pose.

The computer system makes a distance measurement 360 from the imaging device 302 in the second pose to a second point on the second region of the anatomical structure. The distance measurements 360 may indicate a second distance between the imaging device 302 in the second pose and the second point. As with the first pose, the computer system may make determine the distance measurement 360 by triangulating the distance between the imaging device 302 and the second point.

The computer system then uses the kinematics data 350, which indicates the position 352 and the orientation 354 of the imagine device 302, along with the distance measurement 360 to determine coordinates 380 of the second point of the second region. As with the first pose, the computer system may determine the global coordinates of the second point as the coordinates 380 by determining the coordinates that are the distance measurement 360 away from the position 352 in the direction indicated by the orientation 354. The coordinates 380 are displayed on a display and may be expressed as Cartesian coordinates (x₂, y₂, z₂).

FIG. 3C illustrates an example operation 300C performed by the computer system. In the operation 300C, the computer system calculates a distance 390 between the first point and the second point using the coordinates 340 of the first point and the coordinates 380 of the second point. In one example, the distance 390 is a linear distance 392, which may be calculated as the square root of the sum of the squares of the differences between the coordinate pairs of the coordinates 340 and the coordinates 380 (e.g., sqrt ((x₂−x₁)\hat{ }2+(y₂−y₁)\hat{ }2+(z₂−z₁)\hat{ }2). In another example, the distance 390 is a curved distance 392 on the surface of the anatomical structure. The curved distance 392 may be measured from the first point (coordinates 340) to the second point (coordinates 380) along the surface of the anatomical structure. The curved distance 392 may also be referred to as a surface distance.

In some embodiments, an operator of the computer system uses the imaging device 302 to indicate the first point and the second point to the computer system. For example, the operator may adjust the pose of the imaging device 302 to direct the imaging device 302 at the first point or the second point. In this manner, the operator tags the first point or the second point for the computer system using the imaging device 302. Tagging the first point or the second point may be separate from and independent of other control features provided by the system 100, the system 200, or the computer system. For example, tagging the first point or the second point may be separate and independent of a virtual control mode in which the computer system allows the operator to move a virtual cursor to interact with displayed user interface elements. As another example, tagging the first point or the second point may be separate and independent of moving a surgical instrument using the computer system.

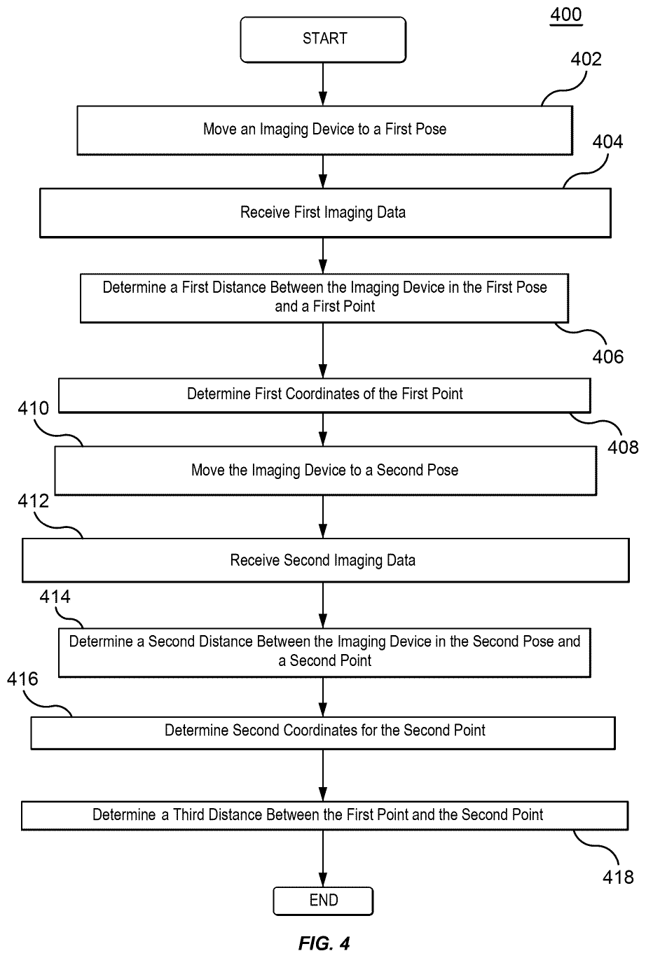

FIG. 4 is a flowchart of an example method 400 performed by the system 100 of FIG. 1A or the system 200 of FIG. 2A. In particular embodiments, the computer system performs the method 400. By performing the method 400, the computer system implements certain features that assist the user to measure a distance between any two points in a 3D space. These features improve the accuracy of the distance measurement, which may improve safety and efficacy of a medical procedure.

In block 402, the computer system moves the imaging device 302 to a first pose. The imaging device 302 may include cameras 304. For example, the imaging device 302 may include a left camera and a right camera that form a stereo camera. These cameras 304 are positioned to capture images or video from slightly different perspectives.

In block 404, the computer system receives the first imaging data 330 captured using the imaging device 302 in the first pose. The first imaging data 330 may include videos 306 captured by the cameras 304 when the imaging device 302 is in the first pose. The computer system monitors or tracks kinematics data 310 that indicates the first pose (e.g., the position 312 and the orientation 314) of the imaging device 302. Generally, the computer system sets the first pose of the imaging device 302 so that the imaging device 302 is positioned close to (e.g., within ten centimeters or five centimeters of) a first point on the first region of the anatomical structure to capture the imaging data 330.

In block 406, the computer system determines a first distance between the imaging device 302 in the first pose and the first point. As an example, the computer system may use the imaging data 330 and a distance between the cameras 304 on the imaging device 302 to triangulate the distance between the imaging device 302 and the first point.

In block 408, the computer system determines the coordinates 340 for the first point. The computer system may use the first pose and the first distance to determine the coordinates 340, which may be expressed as Cartesian coordinates (x₁, y₁, z₁). For example, the computer system may determine the coordinates that are the first distance away from the position 312 in the direction indicated by the orientation 314.

In block 410, the computer system moves the imaging device 302 to a second pose. The second pose is different from the first pose, and when the imaging device 302 is moved to the second pose, the first point may fall out of view from the cameras 304. The imaging device 302 may be moved from the first pose to the second pose by a repositionable structure, such as the manipulator arms 112-1 through 112-4 described in FIG. 1A.

In block 412, the computer system receives second imaging data 370 captured using the imaging device 302 in the second pose. The second imaging data 370 may include videos 306 captured by the cameras 304 when the imaging device 302 is in the second pose. The computer system monitors or tracks the kinematics data 350 that indicates the second pose (e.g., the position 352 and the orientation 354) of the imaging device 302. Generally, the computer system sets the second pose of the imaging device 302 so that the imaging device 302 is close to (e.g., within ten centimeters or five centimeters of) a second point on the second region of the anatomical structure.

In block 414, the computer system determines a second distance between the imaging device 302 in the second pose and the second point. The computer system may use the imaging data 370 and the distance between the cameras 304 on the imaging device 302 to triangulate the distance between the imaging device 302 and the second point.

In block 416, the computer system determines the coordinates 380 for the second point. The computer system may use the second pose and the second distance to determine the coordinates 380, which may be expressed as Cartesian coordinates (x₂, y₂, z₂). For example, the computer system may determine the coordinates that are the second distance away from the position 352 in the direction indicated by the orientation 354.

In block 418, the computer system determines a third distance between the first point and the second point. The computer system may determine the third distance using the coordinates 340 and the coordinates 380. In one example, the computer system calculates the third distance as a liner distance. In another example, the computer system determines the third distance as a distance on the surface of the anatomical structure, e.g., a curved distance. The computer system may display the third distance on the surface of the anatomical structure in a video 306, and the computer system may update the third distance based on various movements of the imaging device 302 and/or of the anatomical structure itself.

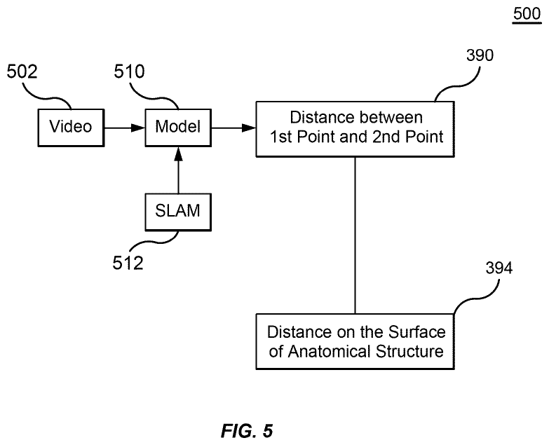

FIG. 5 illustrates an example operation 500 performed by the computer system. Generally, the computer system performs the operation 500 to determine a distance on the surface of the anatomical structure between two points on the surface of the anatomical structure. The computer system uses a simultaneous localization and mapping (SLAM) process to generate a model of the anatomical structure. The computer system then uses the model to determine the distance 394 along the surface of the anatomical structure.

The computer system receives a video 502 of the anatomical structure. The video 502 may be captured by one or more cameras 304 of the imaging device 302. The computer system uses a SLAM process 512 to generate a model 510 of the anatomical structure from the video 502. The model 510 effectively serves as a virtual map of the anatomical structure. SLAM allows the computer system to map the environment in the video 502 while determining the position of the camera 304 capturing the video 502 within that environment. The computer system may localize the camera 304 in the environment using the kinematics data 310 and 350. By localizing the camera 304, the computer system may determine a position and/or orientation of the camera 304 within the environment. The computer system may then present the map and the position and/or orientation of the camera 304 on a display, which provides a more expansive view of the imaging device 302 relative to the surface of the anatomical structure.

During the SLAM process 512, the computer system may stitch together various frames of the video 502 to form the virtual map of the anatomical structure. The computer system may also add virtual geometry to the virtual map. When movement occurs, the computer system may compare the changed view in the video to the virtual map of the anatomical structure to determine the movement or the updated pose of the camera. As a result, the model 510 maps the surface of the anatomical structure.

The computer system may use the model 510 when determining the distance 394 along the surface of the anatomical structure between the first point and the second point. For example, the computer system may have determined the coordinates 340 and 380 of the first and second points. The computer system may then determine a curve along the surface of the anatomical structure in the model 510 between the two coordinates 340 and 380. The computer system may then determine the length of the curve as the distance 394 along the surface of the anatomical structure between the first and second points.

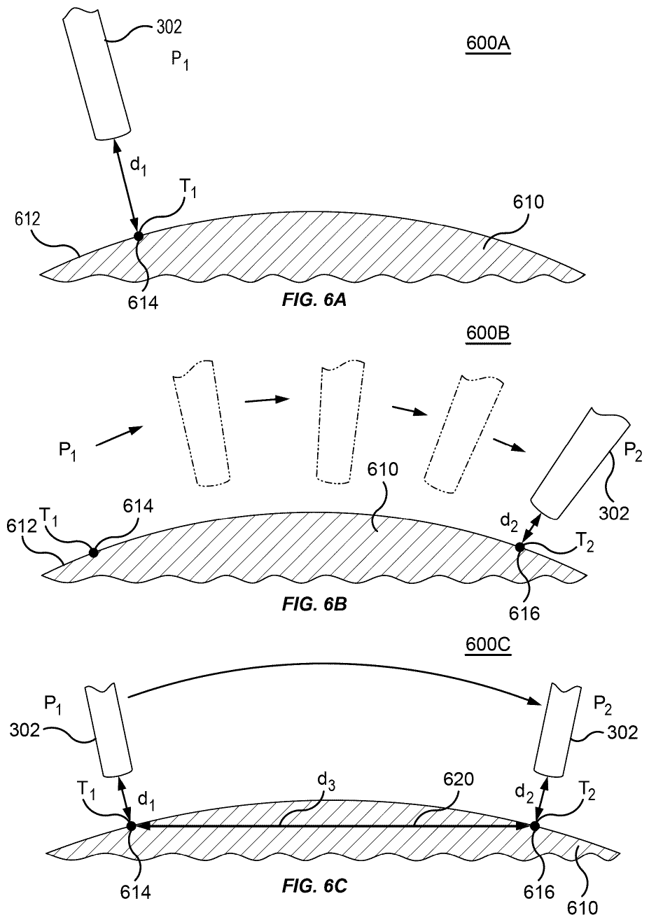

FIGS. 6A through 6C illustrate example operations 600A, 600B, and 600C performed by the computer system. Generally, the computer system moves the imaging device 302 to determine coordinates of the first point of the first region and coordinates of the second point of the second region to determine a third distance between the first point and the second point by moving the imaging device along the surface of the anatomical structure.

FIG. 6A shows the imaging device 302 at a first pose P₁, which may include the position and orientation of the imaging device 302. At the first pose P₁, the imaging device 302 captures first imaging data on the surface 612 of the anatomical structure 610. The computer system uses the first imaging data to triangulate a first distance d₁ between the imaging device 302 and a first point 614 on the surface 612. The first distance d₁ may be less than ten centimeters or five centimeters. The first distance d₁ may be determined based on an axis of the imaging device 302. For example, the first distance d₁ may be along a viewing axis of one of the cameras of the imaging device 302 in the first pose P₁. As another example, the distance d₁ may be along a combination (e.g., resultant sum) of the axes of the cameras of the imaging device 302 in the first pose P₁. Based on the first pose P₁ and the first distance d₁, the computer system determines first coordinates of the first point 614 on the surface 612. The first coordinates may be represented as Cartesian coordinates T₁ (x₁, y₁, z₁), which may be the global coordinates of the first point 614.

FIG. 6B shows the imaging device 302 moved from the first pose P₁ to a second pose P₂. At the second pose P₂, the imaging device 302 captures second imaging data on the surface 612 of the anatomical structure 610. The first point 614 may fall out of view when the imaging device 302 is moved to the second pose P₂. The computer system uses the second imaging data to triangulate a second distance d₂ between the imaging device 302 and a second point 616 on the surface 612. The second distance d₂ may be less than ten centimeters or five centimeters. The second distance d₂ may be determined based on an axis of the imaging device 302. For example, the first distance d₂ may be along a viewing axis of one of the cameras of the imaging device 302 in the second pose P₂. As another example, the distance d₂ may be along a combination (e.g., resultant sum) of the axes of the cameras of the imaging device 302 in the second pose P₂. Based on the second pose P₂ and the second distance d₂, the computer system determines second coordinates of the second point 616 on the surface 612. The second coordinates may be represented as Cartesian coordinates T₂ (x₂, y₂, z₂), which may be the global coordinates of the second point 616.

FIG. 6C shows the computer system determining a distance 620 between the first point 614 and the second point 616. The distance 620 may be a linear distance calculated directly from the coordinates T₁ and T₂. Alternatively or additionally, the distance 620 may be the length of a curve along the surface 612 between the first point 614 and the second point 616.

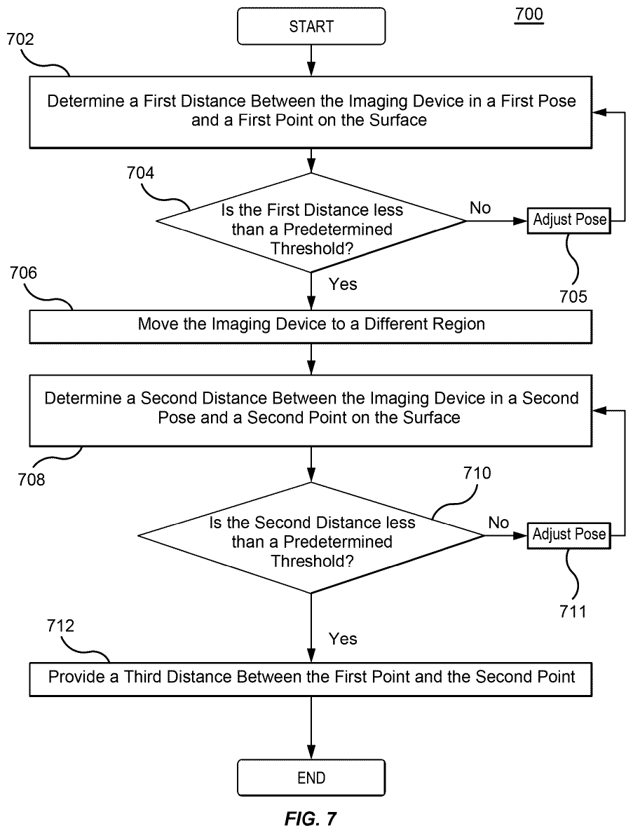

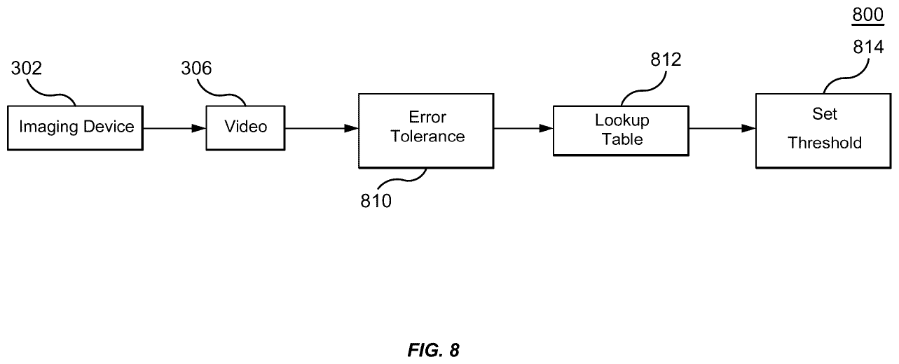

FIG. 7 is a flowchart of an example method 700 performed in the system 100 of FIG. 1A or the system 200 of FIG. 2A. In particular embodiments, the computer system performs the method 700. By performing the method 700, the computer system implements certain features that assist the user to measure a distance between two points. For example, the computer system determines whether triangulated distances between the imaging device 302 and the points are below particular thresholds, which may improve the accuracy of these distance measurements and the accuracy of the determined distance between the two points.

In block 702, the computer system determines a first distance between the imaging device 302 in a first pose and a first point on the surface. For example, the computer system may use a video captured by the imaging device 302 to triangulate the first distance.

In block 704, the computer system determines whether the first distance is less than a predetermined threshold (e.g., ten centimeters or five centimeters). If the first distance exceeds the threshold, the computer system adjusts the pose of the imaging device 302 so that the imaging device 302 is moved closer to the first point in block 705. The computer system then returns to block 702 to determine the first distance.

If the first distance does not exceed the threshold, in block 706, the computer system moves the imaging device 302 to a different region on the surface of the anatomical structure such that the imaging device 302 is in a second pose. Moving the imaging device 302 may cause the first point to fall out of view. The imaging device 302 may be moved from the first pose to the second pose by a repositionable structure, such as the manipulator arms 112-1 through 112-4 described in FIG. 1A.

In block 708, the computer system determines a second distance between the imaging device 302 in the second pose and a second point on the surface. The computer system may use a video captured by the imaging device 302 to triangulate the second distance.

In block 710, the computer system determines whether the second distance is less than a predetermined threshold (e.g., ten centimeters or five centimeters). If the second distance exceeds the threshold, the computer system adjusts the pose of the imaging device 302 such that the imaging device 302 is moved closer to the second point in block 711. The computer system then returns to block 708 to determine the second distance.

If the second distance does not exceed the threshold, in block 712, the computer system determines a third distance between the first point and the second point.

For example, the computer system may determine the global coordinates for the first point by determining the coordinates that are the first distance away from the coordinates of the imaging device 302 in the first pose in the direction indicated by the first pose. The computer system may also determine the global coordinates for the second point by determining the coordinates that are the second distance away from the coordinates of the imaging device 302 in the second pose in the direction indicated by the second pose. The computer system then determines the third distance between the first point and the second point using their global coordinates.

In one example, the computer system determines the threshold based on at least one of a depth measurement at the first point, a depth measurement at the second point, or kinematics errors. The triangulation technique uses a baseline (center point) distance between the left camera and the right camera of the imaging device and the difference in the position of a first point between images from the left camera and the right camera to determine the position of the first point and the distance between the first point and the imaging device. Similarly, the triangulation technique uses a baseline (center point) distance between the left camera and the right camera of the endoscope and the difference in the position of a second point between images from the left camera and the right camera to determine the position of the second point and the distance between the second point and the imaging device. The baseline distance between the left camera and the right camera may be small, which results in the determined distances between the first and second points and the imaging device being more inaccurate the further away the first and second points are from the imaging device. Moving the imaging device closer to the first point or the second point may improve the accuracy of the distance measurement.

The computer system may set the threshold based on kinematics errors. Kinematics errors may relate to inaccuracies or deviations of movements of the imaging device 302 during a medical procedure. Kinematics errors may occur if there are discrepancies between the surgeon's input or instructions and the movement of the imaging device 302. In one example, a kinematics error may relate to a calibration issue. If the threshold is set to ten centimeters and the computer system detects a calibration issue affecting distance measurement accuracy to the first point or the second point, a notification or warning may be provided to the user to move the imaging device 302 to less than ten centimeters from the surface of the anatomical structure (e.g., set the threshold to five centimeters) to avoid the detected calibration issue. As such, detection of kinematics errors may cause the user to move the imaging device 302 closer to the first point and/or the second point.

The computer system may set the threshold so that the distances between the first and second points and the imaging device 302 are accurate. The threshold indicates to the user how far the imaging device may be positioned from the first and second points. The threshold indication allows the user to make adjustments to the positioning of the imaging device 302 (e.g., to move the imaging device 302 closer to or farther from the first and second points) to obtain more accurate distance measurements. The threshold may be based on distance measurements. The computer system measures a distance from the first point to the left camera and measures a distance from the first point to the right camera. If the difference between the two measured distances is too large (e.g., exceeds a difference threshold), then the difference may indicate inaccuracy in the distance measurements. In response, the computer system may reduce the threshold to indicate that the imaging device 302 should be moved closer to the first point to improve the accuracy of the distance measurements.

In another example, instead of using triangulation techniques, the computer system uses an error tolerance and lookup table to set the thresholds, as described below with reference to FIG. 8.

FIG. 8 is illustrates an example operation 800 performed by the computer system. Generally, the computer system may use error tolerance and lookup tables (LUTs) to assist in setting first and second thresholds.

The computer system captures a video 306 from the two cameras of the imaging device 302 and receives an error tolerance 810 from a user. The error tolerance 810 indicates an amount of error in the distance measurement that the user or the surgical procedure can tolerate, e.g., for safety reasons. The computer system then references a lookup table (LUT) 812 to determine the threshold. The LUT 812 may indicate various distances between the imaging device 302 and the point and the errors that can be introduced in the distance measurement at those distances. By referencing into the LUT 812 using the error tolerance 810, the computer system may determine the maximum distance in the LUT 812 that produces errors within the error tolerance 810. For example, the computer system may determine the error in the LUT 812 that comes closest to the error tolerance 810 without exceeding the error tolerance 810. The computer system then determines the distance mapped to that error in the LUT 812. The computer system sets the threshold in block 814 as this distance. In this manner, the computer system sets the threshold for the distance between the imaging device 302 and the point using the error tolerance 810 and the LUT 812.

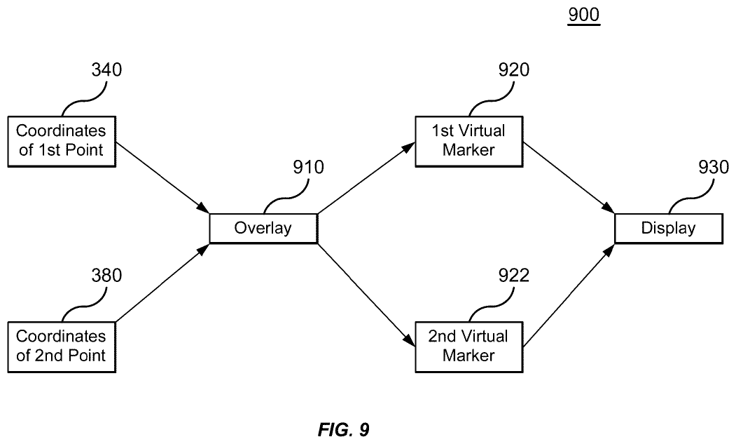

FIG. 9 illustrates an example operation 900 performed by the computer system. Generally, the computer system displays virtual markers to indicate the first point and the second point.

The computer system determines the coordinates 340 of the first point and the coordinates 380 of the second point. The computer system then generates an overlay 910 that includes virtual markers 920 and 922. The virtual marker 920 may indicate the first point, and the virtual marker 922 may indicate the second point. The virtual markers 920 and 922 may be arranged in the overlay 910 according to the coordinates 340 and 380 such that the virtual markers 920 and 922 are positioned over the first point and the second point when the computer system positions the overlay 910 onto a video of the anatomic structure on a display 930.

The first and second virtual markers 920 and 922 may have various presentation properties including, for example, shape, color, size, translucency, surface patterns, text, any other presentation property, and/or a combination thereof. Such presentation properties of the virtual makers 920 and 922 may be used to indicate a direction (e.g., front or back) and an orientation. In an example, the first and second virtual markers 920 and 922 may be shaped as cylinders and may not indicate a direction or an orientation. In another example, the first and second virtual markers 920 and 922 may be shaped as cones and may use a color change, a pattern, a symbol, a text, and/or a combination thereof to indicate a direction and/or an orientation associated with the first and second virtual markers 920 and 922. The first and second virtual markers 920 and 922 may include any shapes. For example, the first and second virtual markers 920 and 922 may include a one-dimensional shape (e.g., a straight line), a two-dimensional shape (e.g., a triangle, a square, a rectangle, a circle, an oval), and/or a three-dimensional shape (e.g., a cylinder, a pyramid, a prism, a cube, a rectangular prism).

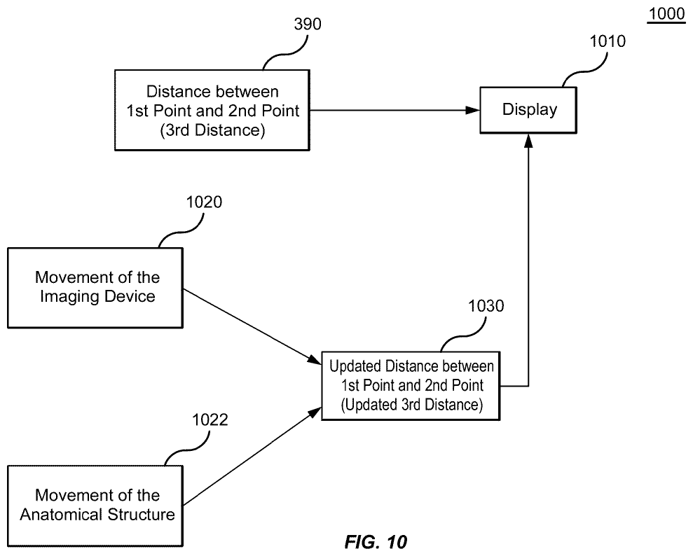

FIG. 10 illustrates an example operation 1000 performed by the computer system. Generally, the computer system displays the determined distance 390 between the first point and the second point on a display 1010.

This distance 390 may be determined according to the operations 300A, 300B, and 300C shown in FIGS. 3A, 3B, and 3C. The computer system also updates the distance 390 when there are movements (e.g., movements of the imaging device 302 or movements of the anatomical structure).

The computer system detects one or more of movement 1020 of the imaging device 302 or movement 1022 of the anatomical structure. The computer system may detect the movement 1020 and/or the movement 1022 by analyzing the frames of a video of the anatomical structure. Differences in the frames may indicate movement. When all the pixels move between subsequent frames, the computer system may detect movement 1020 of the imaging device 302. When a subset of the pixels move between subsequent frames, the computer system may detect movement 1022 of the anatomical structure. In some embodiments, the computer system detects movement 1020 of the imaging device 302 by monitoring or tracking kinematics data for the imaging device 302. When the kinematics data indicates a change in the pose of the imaging device 302, the computer system may analyze the change in the kinematics data to determine the movement 1020 of the imaging device 302.

The computer system may update the distance 390 to produce the updated distance 1030 when movement 1020 of the imaging device 302 occurs. For example, the movement 1020 of the imaging device 302 may change the pose of the imaging device 302 but still keep the imaging device 302 close to the first point or the second point. The computer system may then re-triangulate the distance between the imaging device 302 and the first point or the second point and re-determine the global coordinates for the first point or the second point using the re-triangulated distance and the new pose for the imaging device 302. The computer system then uses the re-determined global coordinates for the first point or the second point when updating the distance 390. In some embodiments, the computer system may combine (e.g., average) the determined global coordinates for the first point or the second point, and use the combined global coordinates as the global coordinates for the first point or the second point. The computer system then updates the distance 390 using the combined global coordinates.

The computer system may update the distance 390 to produce the updated distance 1030 when movement 1022 of the anatomical structure occurs. For example, the anatomical structure may change in position or shape as part of normal physiological functions. A digestive organ, such as the stomach, undergoes coordinated contractions known as peristalsis. Other organs may move as a result of pressure changes in the thoracic cavity during breathing. These changes in position or shape may change the distance between the first point and the second point along the surface of the anatomical structure. When the computer system detects the movement 1022, the computer system may use a SLAM process to update a model of the anatomical structure that serves as a virtual map of the anatomical structure. The computer system may then update distance 390 between the first point and the second point along the surface of the anatomical structure using the updated model.

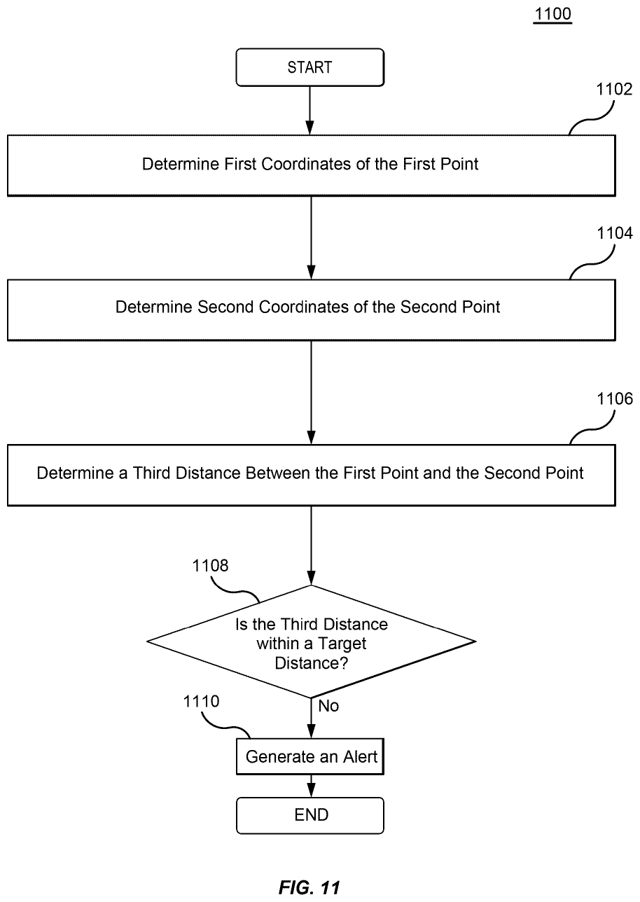

FIG. 11 is a flowchart of an example method 1100 performed in the system 100 of FIG. 1A or the system 200 of FIG. 2A. In particular embodiments, the computer system performs the method 1100. By performing the method 1100, the computer system uses the distance between the first point and the second point to determine whether there is sufficient space to safely perform a medical procedure.

In block 1102, the computer system determines the first coordinates 340 of the first point on the surface of the anatomical structure. The coordinates 340 may be determined according to the operation 300A shown in FIG. 3A.

In block 1104, the computer system determines the second coordinates 380 of the second point on the surface of the anatomical structure. The coordinates 380 may be determined according to the operation 300B shown in FIG. 3B.

In block 1106, the distance 390 between the first point and the second point is determined based on the coordinates 340 for the first point and the coordinates 380 for the second point. The distance 390 may be determined according to the operation 300C shown in FIG. 3C.

In block 1108, the computer system determines whether the distance 390 falls below a target distance. The target distance may ensure that there is sufficient space between the first point and the second point to safely perform a medical procedure (e.g., a surgery or an incision). If the distance 390 falls below the target distance, then the computer system generates an alert in block 1110 to indicate that there is not sufficient space to perform the medical procedure. If the distance exceeds the target distance, then the computer system outputs the distance 390 (e.g., displays the distance 390) to assist the medical procedure.

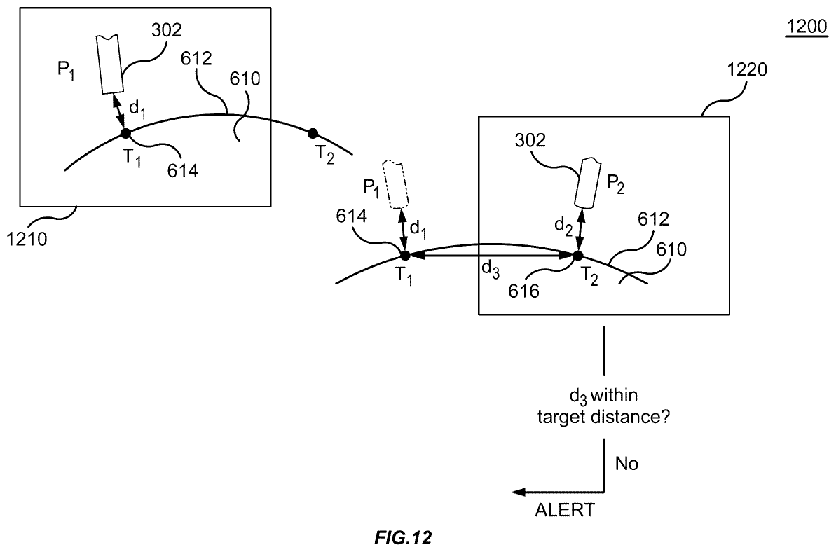

FIG. 12 illustrates an example operation 1200 performed by the computer system. Generally, the computer system determines a distance between the first point 614 and the second point 616, and generates an alert if that distance falls below a threshold.

In a first step 1210, the computer system sets the imaging device 302 at a first pose P₁. In the first pose P₁, the imaging device 302 is close to the surface 612 of the anatomical structure 610. The computer system determines (e.g., using triangulation) a first distance d₁ between the imaging device 302 in the first pose and the first point 614 on the surface 612. Based on the first pose and the first distance, the computer system determines first coordinates T₁ for the first point 614. In one example, the first distance d₁ may be less than ten centimeters. In other examples, the distance d₁ may be less than five centimeters.

In a second step 1220, the computer system moves the imaging device 302 to a second pose P₂ to capture second imaging data showing a second region of the surface 612 of the anatomical structure 610. Moving the imaging device 302 may cause the first point 614 to fall out of view of the imaging device 302. The computer system determines a second distance d₂ between the imaging device 302 in the second pose and the second point 616 on the surface 612. Based on the second pose P₂ and the second distance d₂, the computer system determines second coordinates T₂ for the second point 616. In one example, the second distance d₂ may be less than ten centimeters. In other examples, the distance d₂ may be less than five centimeters. Moving the stereo camera of the imaging device 302 closer to the first point 614 or the second point 616 may improve the accuracy of the distance measurement. Determining correct distances between the first and second points 614 and 616 and the imaging device 302 may be accomplished by setting distance thresholds. The computer system may set the thresholds so that the distances between the first and second points 614 and 616 and the imaging device 302 are accurate. The thresholds indicate to the user how far the imaging device 302 may be from the first and second points 614 and 616. The thresholds allow the user to make adjustments to the positioning of the imaging device 302 (e.g., to move the imaging device 302 closer to or farther from the first and second points 614 and 616) to obtain more accurate distance measurements.

The computer system determines a third distance d₃ between the first point 614 and the second point 616 using the first coordinates T₁ and the second coordinates T₂. The computer system then determines whether the third distance d₃ falls below a target distance. If the third distance d₃ falls below the target distance, the computer system generates an alert to indicate that the third distance d₃ falls below the target distance. An operator of the computer system may then select a different first point 614 and/or a different second point 616, which may increase the third distance d₃ above the threshold.

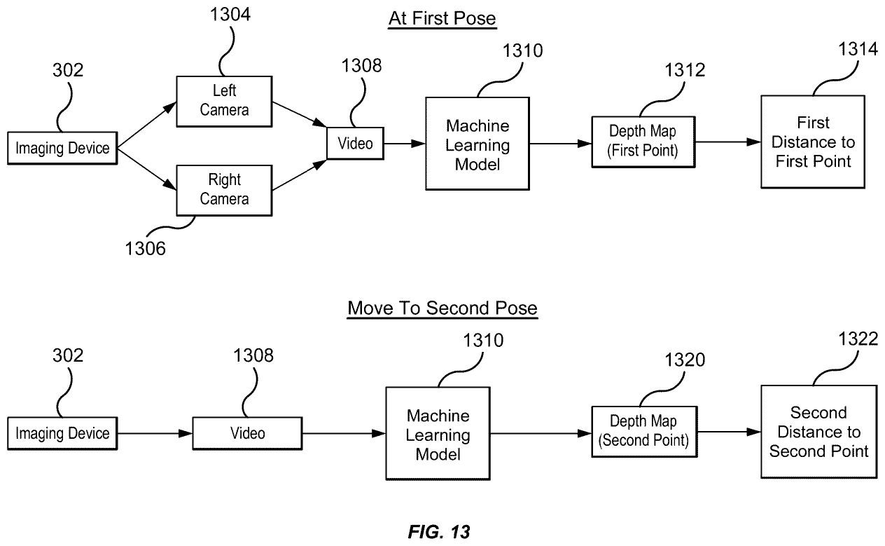

FIG. 13 illustrates an example operation 1300 performed by the computer system. Generally, the computer system may use machine learning (e.g., neural networks) to assist in determining the distance between two points.

An imaging device 302 includes a left camera 1304 and a right camera 1306. A video 1308 is captured by the left camera 1304 and the right camera 1306 when the imaging device 302 is in a first pose. A first depth map 1312 may be generated using a machine learning model 1310 (e.g., a neural network). This data may be used to determine the first distance 1314 to the first point. The imaging device 302 is then moved to a second pose. A video 1308 is captured at the second pose. A second depth map 1320 may be generated using the machine learning model 1310. This data may be used to determine the second distance 1322 to the second point.

In one example, the machine learning model 1310 may include one or more neural networks. The neural networks may have been trained by reviewing videos of various surgical procedures. These videos may show regions of organs or anatomical structures that have been operated on. These videos may further show procedures performed on these regions along with the results. By analyzing these videos, the neural networks may learn how to determine the distances to points in the videos, which is used when generating depth maps. The neural networks may then be used during subsequent medical procedures to guide or assist the procedures by analyzing the videos 1308 to generate the depth maps 1312 and 1320 that indicate the depths of or distances to points shown in the videos 1308.

In summary, the present disclosure describes a medical system that measures a distance between two points in a surgical space by leveraging kinematics data that describes the pose (e.g., position and orientation) of an endoscope. Generally, the system adjusts the pose of the endoscope so that the endoscope is close to a first point to triangulate the distance between the first point and the endoscope. The kinematics data indicates the pose of the endoscope, and so the kinematics data may be used with the determined distance between the first point and the endoscope to determine global coordinates for the first point. The system then adjusts the pose of the endoscope so that the endoscope is close to a second point to triangulate the distance between the second point and the endoscope. The kinematics data indicates the new pose of the endoscope, and so the kinematics data may be used with the determined distance between the second point and the endoscope to determine global coordinates for the second point. Adjusting the pose of the endoscope so that the endoscope is close to the second point may cause the first point to fall out of view. Nevertheless, because the kinematics data indicates the poses of the endoscope, it is possible to use the kinematics data to determine the global coordinates of the first point and the second point. These global coordinates are then used to determine a distance between the first point and the second point.

This description and the accompanying drawings that illustrate aspects, embodiments, or modules should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure other features. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to be limiting. For example, spatially relative terms-such as “beneath”, “below”, “lower”, “above”, “upper”, “proximal”, “distal”, and the like may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, or module may, whenever practical, be included in other embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, or application may be incorporated into other embodiments, or aspects unless specifically described otherwise, unless the one or more elements would make an embodiment or embodiments non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system, such as the DA VINCI SURGICAL SYSTEM or ION SYSTEM commercialized by Intuitive Surgical, Inc. of Sunnyvale, California. Knowledgeable persons will understand, however, that aspects disclosed herein may be embodied and implemented in various ways, including robotic and, if applicable, non-robotic embodiments. Techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the disclosure should be limited only by the following claims, and it is appropriate that the claims be construed broadly and, in a manner, consistent with the scope of the embodiments disclosed herein.

What is claimed is:

1. A system for measuring a distance between two points in a surgical space, the system comprising:
a memory; and
a controller communicatively coupled to the memory, the controller configured to:
move an imaging device to a first pose to capture first imaging data showing a first region of a surface of an anatomical structure;
determine a first distance between the imaging device in the first pose and a first point on the surface;
determine, based on the first pose and the first distance, first coordinates of the first point on the surface;
move the imaging device to a second pose to capture second imaging data showing a second region of the surface of the anatomical structure;
determine a second distance between the imaging device in the second pose and a second point on the surface;
determine, based on the second pose and the second distance, second coordinates for the second point on the surface; and
based on each of the first and second distances to the first and the second points on the surface, respectively, being within a threshold, determine, based on the first coordinates and the second coordinates, a third distance between the first point and the second point.

2. The system of claim 1, wherein the third distance is a linear distance.

3. The system of claim 1, wherein the third distance is a distance on the surface.

4. The system of claim 1, wherein the controller is further configured to update the third distance based on movement of the surface of the anatomical structure.

5. The system of claim 1, wherein determining the first distance comprises:
determining a distance between a left camera of the imaging device and the first point;
determining a distance between a right camera of the imaging device and the first point; and
determining the first distance based on the distance between the left camera and the first point and the distance between the right camera and the first point.

6. The system of claim 1, wherein the threshold is determined based on at least one of a depth measurement at the first point, a depth measurement at the second point, or kinematics errors.

7. The system of claim 1, wherein the threshold is determined based on a received error tolerance and a lookup table.

8. The system of claim 1, wherein the controller is further configured to determine the first pose and the second pose based on kinematics data associated with the imaging device.

9. The system of claim 1, wherein the controller is further configured to control a repositionable structure to move the imaging device to the first pose and the second pose.

10. The system of claim 1, wherein the controller is further configured to:
present, on a display, a video from the imaging device showing the surface of the anatomical structure; and
add, to the video, a first virtual marker overlaid on the first point; and
add, to the video, a second virtual marker overlaid on the second point.

11. The system of claim 1, wherein the first distance is determined based on an axis of the imaging device.

12. The system of claim 1, wherein, when viewing the second region, the first region is out of view of the imaging device.

13. The system of claim 1, wherein the controller is further configured to present the third distance on a display.

14. The system of claim 1, wherein the controller is further configured to update the third distance based on movement of the imaging device or movement of the anatomical structure.

15. The system of claim 1, wherein the controller is further configured to communicate an alert in response to determining that the third distance reached or fell below a target distance.

16. The system of claim 1, wherein indication of the first point and the second point on the surface of the anatomical structure is independent of entering a virtual mode and moving a virtual cursor.

17. The system of claim 1, wherein indication of the first point and the second point on the surface of the anatomical structure is independent of moving a surgical instrument.

18. The system of claim 1, wherein determining the first distance comprises generating a first depth map of the surface of the anatomical structure based on the first imaging data, wherein the first distance is determined based on the first depth map.

19. A method for measuring a distance between two points in a surgical space, the method comprising:
moving an imaging device to a first pose to capture first imaging data showing a first region of a surface of an anatomical structure;
determining a first distance between the imaging device in the first pose and a first point on the surface;
determining, based on the first pose and the first distance, first coordinates of the first point on the surface;
moving the imaging device to a second pose to capture second imaging data showing a second region of the surface of the anatomical structure;
determining a second distance between the imaging device in the second pose and a second point on the surface;
determining, based on the second pose and the second distance, second coordinates for the second point on the surface; and
based on each of the first and second distances to the first and the second points on the surface, respectively, being within a threshold, determining, based on the first coordinates and the second coordinates, a third distance between the first point and the second point.

20. A non-transitory machine-readable medium storing instructions for measuring a distance between two points in a surgical space, when executed by a processor, cause the processor to:
move an imaging device to a first pose to capture first imaging data showing a first region of a surface of an anatomical structure;
determine a first distance between the imaging device in the first pose and a first point on the surface;
determine, based on the first pose and the first distance, first coordinates of the first point on the surface;
move the imaging device to a second pose to capture second imaging data showing a second region of the surface of the anatomical structure;
determine a second distance between the imaging device in the second pose and a second point on the surface;
determine, based on the second pose and the second distance, second coordinates for the second point on the surface; and
based on each of the first and second distances to the first and the second points on the surface, respectively, being within a threshold, determine, based on the first coordinates and the second coordinates, a third distance between the first point and the second point.
